(19) United States
(12) Patent Application Publication
(10) Pub. No.: US 2013/0158654 A1
(43) Pub. Date: Jun. 20, 2013

(54) HEART VALVE REPLACEMENT CATHETER

(71) Applicant: Boston Scientific Scimed, Inc., Maple Grove, MN (US)

(72) Inventors: Benjamin Sutton, San Jose, CA (US); David J. Paul, Scotts Valley, CA (US)

(73) Assignee: BOSTON SCIENTIFIC SCIMED, INC., Maple Grove, MN (US)

(21) Appl. No.: 13/714,879

(22) Filed: Dec. 14, 2012

Related U.S. Application Data
(60) Provisional application No. 61/577,891, filed on Dec. 20, 2011.

(51) Int. Cl. A61F 2/24

(52) U.S. Cl. CPC A61F 2/2427
USPC 623/2.11

(57) ABSTRACT

The disclosure pertains to catheter-based delivery system for intravascular heart valve replacement and methods of use thereof. In addition to the replacement heart valve and associated positioning and installation components, the delivery catheter of the system includes a deployable filter element, an actuator for the deployable filter element, and a fluid expandable balloon located proximate the distal end of the delivery catheter. The delivery catheter may be capable of carrying out a valvuloplasty and/or a determination of a distance within a patient.

### TECHNICAL FIELD

The present disclosure pertains to medical devices, and methods for manufacturing medical devices. More particularly, the present disclosure pertains to medical devices for delivering a replacement heart valve.

### BACKGROUND

A wide variety of intracorporeal medical devices have been developed for medical use, for example, intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure pertains to a system for delivery of a replacement heart valve comprising a delivery catheter having a proximal end, a distal end, and one or more lumens therebetween; a fluid expandable balloon associated with the delivery catheter, said fluid expandable balloon being located near and proximal the distal end of the delivery catheter; a deployable filter element deployed about the delivery catheter and located downstream of the fluid expandable balloon, wherein the deployable filter element located downstream of the fluid expandable balloon has a first collapsed configuration and a second deployed configuration which filters substantially all blood passing the deployable filter element; an actuator associated with and capable of moving the deployable filter element from first collapsed configuration to a second expanded configuration and from a second expanded configuration to a first collapsed configuration; a replacement heart valve; and components configured and adapted to position, and install a replacement heart valve, wherein at least one lumen of the one or more lumens is an inflation lumen.

This disclosure also pertains to a system and method for percutaneous valvuloplasty and heart valve replacement comprising inserting percutaneously a delivery catheter comprising an elongated tubular element having a proximal end, a distal end, and at least one lumen therebetween, a fluid expandable balloon, a deployable filter element, a replacement heart valve, wherein the deployable filter element is located downstream of the fluid expandable balloon and has a first collapsed configuration and a second deployed configuration which filters substantially all blood passing the deployable filter element; and wherein the delivery catheter further includes components which are configured and adapted to position and install a replacement heart valve; advancing the delivery catheter until the fluid expandable balloon is positioned within a heart valve to be replaced; actuating the deployable filter element from the first collapsed configuration to a second expanded configuration; expanding the fluid expandable balloon within the heart valve to be replaced thereby performing a valvuloplasty; collapsing the fluid expandable balloon; actuating the deployable filter element from the second expanded configuration to the first collapsed configuration thereby containing any collected debris within the deployable filter element; retracting the delivery catheter to remove the fluid expandable balloon from the heart valve to be replaced; advancing the replacement heart valve and components configured and adapted to position and install a replacement heart valve from the distal end of the delivery catheter; installing the replacement heart valve; retracting the components which are configured and adapted to position and install the replacement heart valve within the distal end of the delivery catheter; and withdrawing the delivery catheter.

### BRIEF DESCRIPTION OF DRAWINGS

FIGS. 1A-B illustrate schematically an embodiment of the disclosure.

FIGS. 1C-D illustrate schematically another embodiment of the disclosure.

FIG. 2A illustrates schematically a transverse cross-section of a fluid expandable balloon of the disclosure.

FIG. 2B illustrates schematically another transverse cross-section of a fluid expandable balloon of the disclosure.

FIG. 3 illustrates schematically an embodiment of the invention during deployment of a replacement heart valve.

### DETAILED DESCRIPTION

The following description should be read with reference to the drawings wherein like reference numerals indicate like elements throughout the several views. The drawings, which are not necessarily to scale, are not intended to limit the scope of the claimed invention. The detailed description and drawings illustrate example embodiments of the claimed invention.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments whether or not explicitly described unless clearly stated to the contrary.

When performing a percutaneous heart valve replacement, it is often desirable to first perform a valvuloplasty to prepare the native valve to be replaced to receive a replacement valve to be positioned therewithin. Valvuloplasty may be useful to break and/or remove calcifications, restore flexibility, separate fused commissures, and the like. Such procedures may generate undesirable debris and additional debris may be generated during removal of the valvuloplasty catheter prior to the insertion of the delivery catheter for the replacement heart valve.

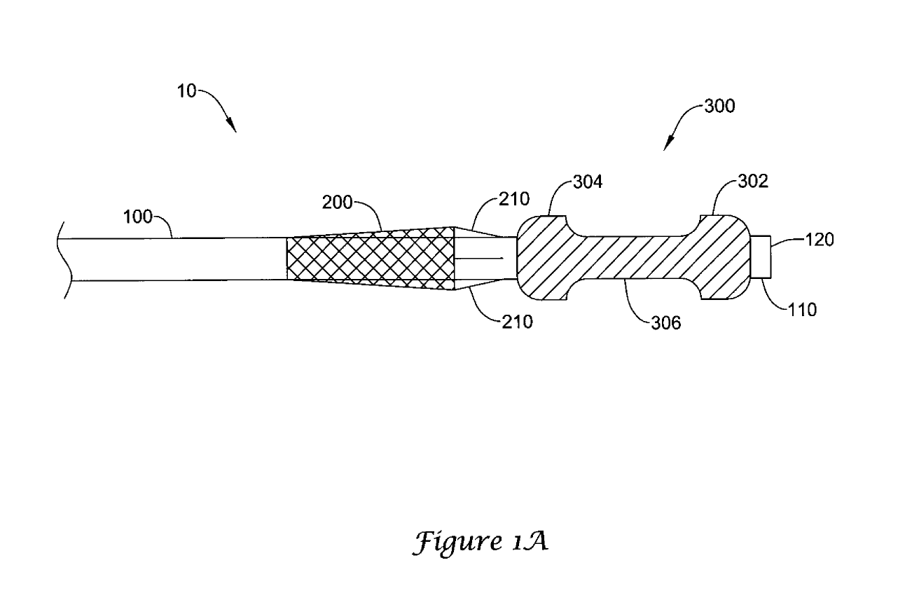

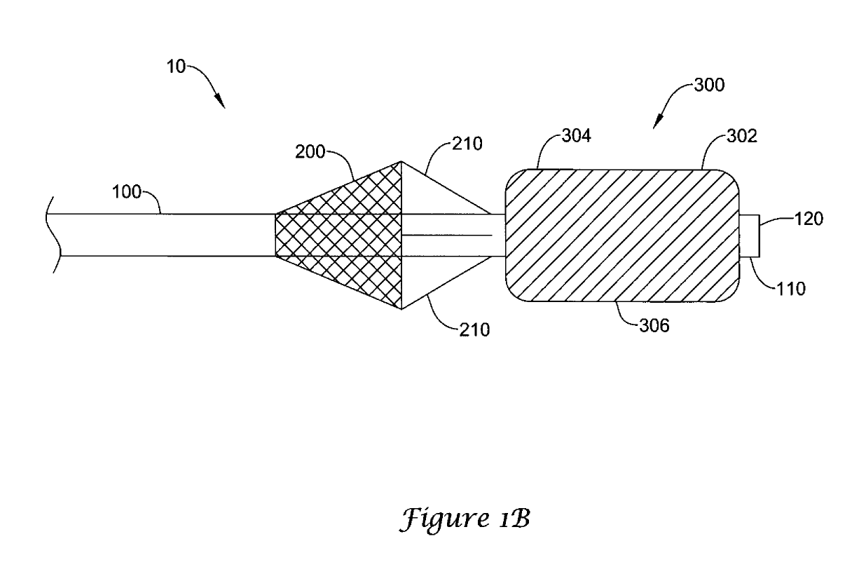

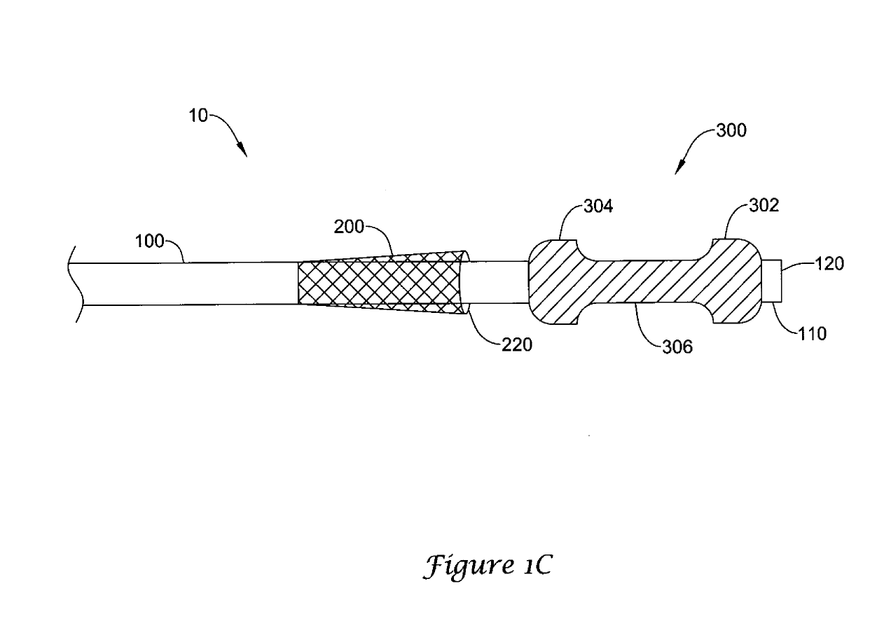

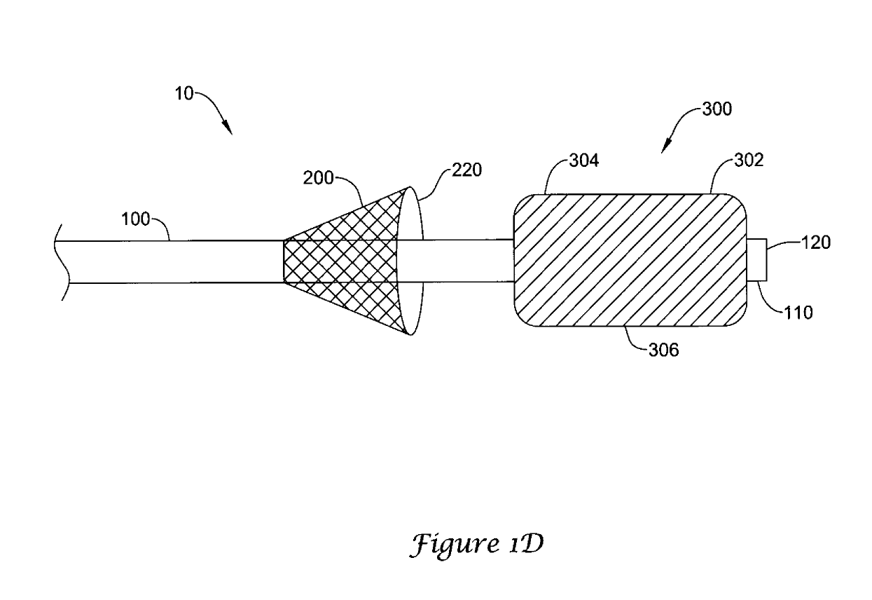

FIGS. 1A-D illustrate somewhat schematically replacement heart valve delivery catheters 100 of a replacement heart valve delivery system 10 which include near and proximal the distal ends 110 thereof, a fluid expandable balloon 300 and a deployable filter element 200. The replacement heart valve delivery catheters 100 are presented in their deployment configurations in FIGS. 1A and 1C and in their filtering and valvuloplasty configurations in FIGS. 1B and 1D. In these non-limiting exemplary configurations, the replacement heart valve and associated positioning and installation components remain retracted within a lumen 120 of the replacement heart valve delivery catheter 100. The replacement heart valve delivery catheter 100 of replacement heart valve delivery system 10 will typically include multiple lumens (not shown in detail) which may be arranged and configured to accommodate various components associated with, for example, the replacement heart valve and various positioning and installation components (not shown) associated therewith, a deployable filter element 200 and its associated actuator(s), such as tethers 210 and/or inflatable ring 220, and/or aspiration ports (not shown), a fluid expandable balloon 300 and its associated inflation/deflation lumen(s) (not shown), and other conventional associated components such as guidewires, sensors, and the like whose presence may be desirable. In the figures, reference numeral 120 should be understood to indicate all of the individual lumens which are necessary to the functioning of a replacement heart valve delivery catheter 100. This schematic representation is not intended to limit the size, length, or relative positions of the various individual lumens or their connections to other components of the replacement heart valve delivery system 10.

Deployable filter element 200 may be selectively actuated between a collapsed configuration, in which it lies substantially along delivery catheter 100 and downstream of fluid expandable balloon 300, and a deployed configuration in which substantially all blood passing the deployable filter element passes through the deployable filter element thereby collecting unwanted debris from the bloodstream. The deployable filter element 200 may be moved between the deployed configuration and the collapsed configuration by an actuator or actuating mechanism, such as tethers 210 and/or inflatable ring 220, as necessary. Although a deployable filter element 200 employing two known actuation mechanisms have been illustrated, it will be appreciated that any of the known deployable filter elements and their associated actuators may be employed. For example, in FIGS. 1A and 1B, a deployable filter element 200 supported by resilient struts (not shown) may be deployed and collapsed by tethers 210. Similarly, the embodiment of FIGS. 1C and 1D may be deployed and collapsed by an inflatable ring 220 positioned at the mouth of the deployable filter element 200. In other embodiments, the deployable filter element may be self-expanding and may be selectively actuated between a first collapsed configuration and a second deployed configuration by a sheath, an advanceable ring, a drawstring, a noose, or other suitable means known in the art.

Delivery catheter 100 of replacement heart valve delivery system 10 also includes a fluid expandable valvuloplasty balloon 300 located at or near distal end 110 of delivery catheter 100. The fluid expandable balloon 300 associated with the delivery catheter 100 is capable of being expanded from a first collapsed configuration to a second expanded configuration by fluid supplied through an inflation lumen of the delivery catheter 100. Additionally, the fluid expandable balloon 300 associated with the delivery catheter 100 is capable of being collapsed from a second expanded configuration to a first collapsed configuration by removal of fluid from the fluid expandable balloon 300 through the inflation lumen of the delivery catheter 100. As illustrated in FIGS. 1A-D, the fluid expandable valvuloplasty balloons 300 may include distal segments 302, intermediate segments 306, and proximal segments 304. In some embodiments, the balloon segments 302 and 304 may have a different mechanical response to fluid pressure applied within the fluid expandable balloon 300. In some embodiments, the distal and proximal segments 302 and 304 of the fluid expandable balloon 300 may be more resistant to radial expansion upon application of fluid pressure to the interior of the fluid expandable balloon 300 than is the intermediate segment 306. In certain embodiments the intermediate segment 306 of the fluid expandable balloon 300 has a mean diameter in an unpressurized state which is less than the mean diameter in an unpressurized state of the distal segment 302 and which is less than the mean diameter in an unpressurized state of the proximal segment 304.

In such embodiments, the distal and proximal segments 302 and 304 of the fluid expandable balloon 300 may tend to stabilize the position of the fluid expandable balloon 300 within the native valve during the valvuloplasty portion of the procedure. In the unpressurized state, such distal and/or proximal segments 302, 304 may partially collapse during insertion through the native valve, whereupon they may elastically rebound to the positions indicated by FIGS. 1A and 1C. In those embodiments, the intermediate segment 306 may be inflated to approximate the diameter of the distal and proximal segments 302, 304, as illustrated in FIGS. 1B and 1D, without significant risk of the distal and proximal segments 302, 304 of the fluid expandable balloon 300 being displaced from their positions on either side of the native valve. While the fluid expandable balloon 300 is held in position by segments 302, 304 or other means, expansion of intermediate segment 306 may effect the valvuloplasty. In certain embodiments, deployable filter element 200, in its deployed configuration, may provide additional longitudinal positional stability to the fluid expandable balloon 300 through its interaction with adjacent body tissue. It will be appreciated that other valvuloplasty balloon configurations known in the art may be employed with the delivery catheter 100 of the disclosure. The illustrated valvuloplasty balloon configurations are intended to be generically exemplary of such balloons and non-limiting.

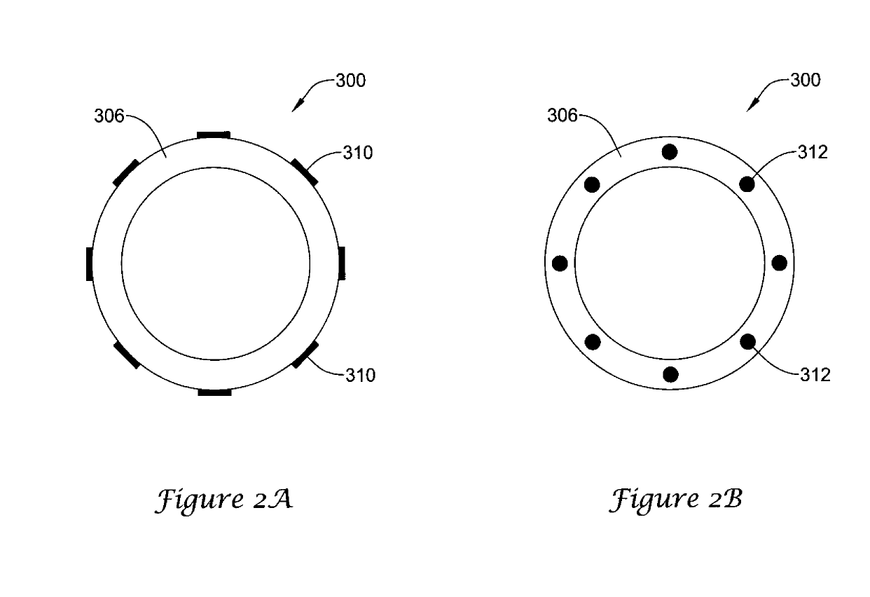

In some embodiments, fluid expandable balloon 300 may also provide an additional function during the valvuloplasty portion of the heart valve replacement procedure by providing a measure of a dimension associated with the native valve. FIGS. 2A and 2B illustrate non-limiting examples of intermediate balloon segments 306 of fluid expandable balloons 300 which comprise at least one measuring element for determining distances within a patient. With such elements, the diameter of the intermediate segment 306 of the fluid expandable balloon 300 may be sensed during the valvuloplasty portion of the heart valve replacement procedure. In those embodiments, for example, the mean diameter of an aperture defined by a dilated native valve to be replaced may be determined by fluoroscopy in combination with radiopaque markers 310, 312 distributed about a circumference of an inflated fluid expandable balloon 300. In some embodiments, markers 310 may be disposed on an interior or an exterior wall of the fluid expandable balloon 300, for example along the exterior of intermediate segment 306. In other embodiments, markers 312 may be disposed within the wall of a fluid expandable balloon 300, for example within the wall of an intermediate segment 306. In yet other embodiments, rather than employing fluoroscopy to visualize the markers, the markers may be visualized by other means such as magnetic resonance imaging (MRI), ultrasound, and the like. Accordingly, depending upon the visualization means to be employed, the markers may comprise metallic deposits, wires, liquid inclusions, and the like. The markers 310, 312 may be somewhat planar, elongated, or substantially spherical beads or droplets.

It will be appreciated that other dimension determining means may also be employed. For example, the fluid expandable balloon may be inflated with a radiopaque fluid contrast medium or an appropriate segment of the fluid expandable balloon may comprise strain measuring sensors and the like. In some embodiments, the mean diameter of an aperture defined by a dilated valve to be replaced may be determined from a combination of a volume of fluid delivered to the fluid expandable balloon and a pressure within the fluid expandable balloon resulting from the delivery of that volume of fluid to the fluid expandable balloon. The relevant dimension may be determined from the previously determined elastic response of the fluid expandable balloon 300 in conjunction with the appearance of inflection points in a plot of pressure within the balloon as a function of the addition of known volumes of inflation fluid to the balloon lumen which may indicate resistance of a segment to further expansion as it fully dilates the native valve.

During the valvuloplasty and/or dimension determination(s) described herein, debris may be released from the native valve. Such debris may include dislodged calcifications and the like. Accordingly it is desirable to position a deployable filter element 200 downstream from the valvuloplasty site during at least the expansion phase of the valvuloplasty. When the deployable filter element 200 is located downstream of the fluid expandable balloon, it may be desirable that it be configured and adapted to be actuated such that it is placed in the second deployed configuration while the fluid expandable balloon 300 is positioned with the intermediate segment 306 generally centered within the dilated native valve to be replaced and while the fluid expandable balloon 300 is first inflated and then deflated thereby allowing collection of debris.

It will be readily appreciated that it also may be desirable to have a deployable filter element 200 deployed downstream during other phases of the heart valve replacement procedure. For example, it may also be desirable for the deployable filter element 200 to be deployed downstream of the distal end of the delivery catheter during the insertion and deployment of the heart valve replacement. Accordingly, it may be desirable for the deployable filter element 200 to be capable of being actuated to assume a partially or completely collapsed configuration following the valvuloplasty phase of the procedure thereby allowing the balloon 300 of delivery catheter 100 to be repositioned proximal of the native valve to be replaced and then to be subsequently redeployed for a period of time necessary to complete the heart valve replacement before being collapsed and withdrawn with the captured debris at the conclusion of the heart valve replacement procedure.

It also will be appreciated that a variety of deployable filter elements 200 and associated actuator(s), such as tethers 210 and/or inflatable ring 220, as well as others, are known and readily adapted for use with a delivery catheter 100. Although the deployable filter elements 200 have been depicted as having a mesh-like filtering body, perforated sheets of metal or polymer may also be employed. In other embodiments (not illustrated), straight, curved, and/or branched struts may provide the debris capturing filter body. In yet other embodiments, the filter element 200 and delivery catheter 100 may include an aspiration lumen (not shown) capable of removing at least some of the collected debris prior to collapse of the filter element 200. Additionally, although a generally symmetrical and conical deployable filter element 200 has been illustrated, it will be appreciated that other forms are also contemplated and may include, for example, generally hemispherical collection bodies, extended tubular filtration bodies, partially everted bodies, and the like which are known in the art, with the selection of a form factor depending upon how far downstream and where within the patient's anatomy the deployable filter element 200 is to be deployed. The selected deployment location may also dictate the radial extent of the deployed configuration relative to the diameter of the deployment catheter.

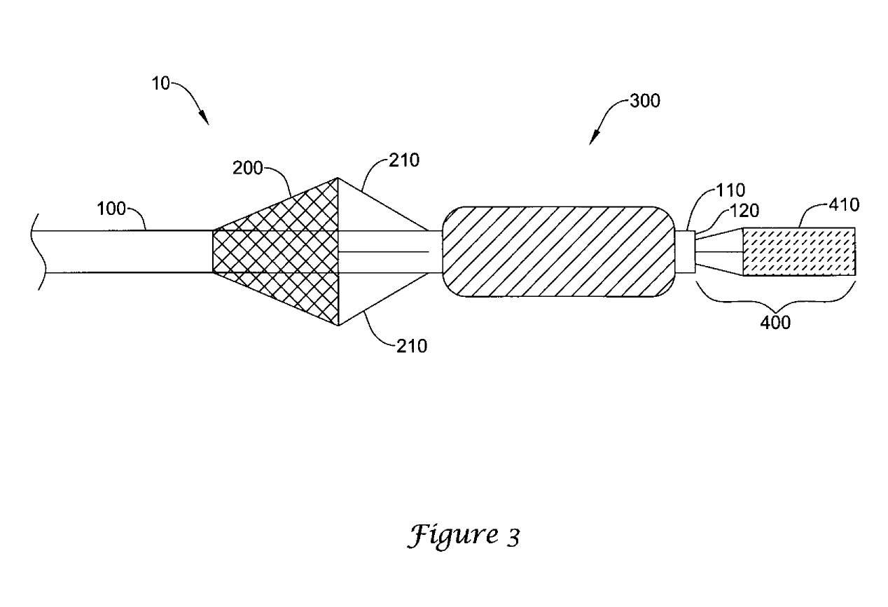

FIG. 3 illustrates the configuration of a delivery catheter 100 at a later stage of the heart valve replacement process. In this configuration, the fluid expandable balloon 300 has been deflated to minimize occlusion of the blood vessel while the deployable filter element 200 has been actuated to assume a second deployed configuration which filters substantially all blood passing the deployable filter element 200. Replacement heart valve 410 and its associated positioning and installation components, collectively indicated by reference numeral 400, have been extended from a lumen 120 of the delivery catheter 100 thereby positioning the replacement heart valve 410 within a lumen defined by the native heart valve (not shown). In the illustrated configuration of FIG. 3, the replacement heart valve 410 has not yet been expanded and installed.

In use, the delivery catheter 100 may be inserted percutaneously in the conventional manner and advanced through the vasculature until fluid expandable balloon 300 is positioned within the native heart valve to be replaced. In some embodiments, the intermediate segment 306 is positioned within the native heart valve while distal and proximal segments 302 and 304 of the fluid expandable balloon 300 are positioned on either side of the native heart valve. It will be appreciated that this description is somewhat dependent on the exemplary fluid expandable balloon configuration illustrated and may be subject to modification when other fluid expandable balloons are employed.

Deployable filter element 200 may be actuated from the first collapsed configuration to a second expanded configuration adapted to capture debris by an appropriate actuator or actuating mechanism, such as tethers 210 or inflatable ring 220. Once the deployable filter element 200 is expanded, fluid expandable balloon 300 may be expanded within the native heart valve to perform a valvuloplasty. In some embodiments, a step of determining at least one distance within a patient may be inserted into the procedure at this point. For example, the position of markers associated with the fluid expandable balloon 300 may be determined by fluoroscopy, MRI, or the like thereby determining the mean diameter of the aperture defined by the dilated native heart valve. In other embodiments, the distance determination may be accomplished by other means discussed herein. The distance determination may serve any of several purposes. For example, the determination of mean diameter of the aperture defined by the dilated native heart valve may be used to confirm that the replacement heart valve 410 being delivered by delivery catheter 100 is appropriately sized for the patient's anatomy before proceeding with the deployment and installation of the replacement heart valve.

Following completion of the valvuloplasty, fluid expandable balloon 300 may be collapsed thereby restoring blood flow through the native heart valve and allowing the fluid inflatable balloon 300 to be repositioned. The deployable filter element 200 may be actuated from the second deployed configuration to the first collapsed configuration thereby containing any collected debris within the deployable filter element 200. In some embodiments, the deployable filter element 200 may only be partially actuated from the second expanded configuration toward the first collapsed configuration to maintain a degree of filtration while allowing the delivery catheter 100 to be retracted to remove the fluid expandable balloon 300 from the native heart valve to be replaced. In certain embodiments, debris may be removed from the deployable filter element by application of a vacuum to an aspiration port (not shown) connected to a lumen 120 of the delivery catheter 100. If debris is to be removed in this manner, it may be removed at any time during the procedure which is consistent with the operation of the delivery catheter 100.

Once the delivery catheter 100 is repositioned, the deployable filter element 200 may be actuated from the first collapsed configuration to a second deployed configuration to resume filtration of the blood stream if desired or may be maintained in the first collapsed configuration during the installation of the replacement heart valve 410.

The replacement heart valve 410 and its associated components 400 which are configured and adapted to position and install the replacement heart valve may be advanced from a lumen 120 of delivery catheter 100 and positioned within the native heart valve to be replaced. Installation of the replacement heart valve 410 may then proceed in a manner consistent with the design of the replacement heart valve 410 and its associated components 400. Following installation of the replacement heart valve 410, the components 400 which positioned and installed the replacement heart valve 410 may be retracted within a lumen 120 at the distal end 110 of delivery catheter 100. If filter element 200 remains in a second deployed configuration, it may be actuated to assume the first collapsed configuration thereby containing any collected debris within the deployable filter element. Having completed the installation of replacement heart valve 410, an operator may then withdraw the delivery catheter from the patient.

Although the illustrative examples described above relate to replacement of a native heart valve, it is also contemplated that the apparatus described may also be used to install or replace other valves within body lumens. In such an embodiment, the valve and associated valve positioning and installation components may be altered as appropriate. It may also be desirable to alter the size and location of the fluid expandable balloon and the deployable filter element.

Various modifications and alterations of this invention will become apparent to those skilled in the art without departing from the scope and principles of this invention, and it should be understood that this invention is not to be unduly limited to the illustrative embodiments set forth hereinabove. All publications and patents are herein incorporated by reference to the same extent as if each individual publication or patent was specifically and individually indicated to be incorporated by reference.

What is claimed is:

1. A system for delivery of a replacement heart valve comprising:
a delivery catheter having a proximal end, a distal end, and one or more lumens therebetween;
a fluid expandable balloon associated with the delivery catheter, said fluid expandable balloon being located near and proximal the distal end of the delivery catheter;
a deployable filter element deployed about the delivery catheter and located downstream of the fluid expandable balloon,
wherein the deployable filter element located downstream of the fluid expandable balloon has a first collapsed configuration and a second deployed configuration which filters substantially all blood passing the deployable filter element;
an actuator associated with and capable of moving the deployable filter element from first collapsed configuration to a second expanded configuration and from a second expanded configuration to a first collapsed configuration;
a replacement heart valve; and
components configured and adapted to position, and install a replacement heart valve,
wherein at least one lumen of the one or more lumens is an inflation lumen.

2. The delivery catheter of claim 1, wherein the fluid expandable balloon associated with the delivery catheter is capable of being expanded from a first collapsed configuration to a second expanded configuration by fluid supplied through the inflation lumen of the delivery catheter.

3. The delivery catheter of any of claims 1 and 2, wherein the fluid expandable balloon associated with the delivery catheter is capable of being collapsed from a second expanded configuration to a first collapsed configuration by removal of fluid from the fluid expandable balloon through the inflation lumen of the delivery catheter.

4. The delivery catheter of any of claims 1 through 3, wherein the fluid expandable balloon comprises at least one measuring element for determining a distance within a patient.

5. The delivery catheter of any of claims 1 through 4, wherein the fluid expandable balloon comprises a distal segment, an intermediate segment, and a proximal segment.

6. The delivery catheter of claim 5, wherein the distal segment and the proximal segment of the fluid expandable balloon are more resistant to radial expansion upon application of fluid pressure to the interior of the fluid expandable balloon than is the intermediate segment.

7. The delivery catheter of any of claims 5 and 6, wherein the intermediate segment of the fluid expandable balloon has a mean diameter in an unpressurized state which is less than the mean diameter in an unpressurized state of the distal segment and is less than the mean diameter in an unpressurized state of the proximal segment.

8. The delivery catheter of any of claims 4 through 7, wherein the at least one measuring element for determining distances within the patient comprises the intermediate segment of the fluid expandable balloon.

9. The delivery catheter of claim 8, wherein the mean diameter of an aperture defined by a dilated valve to be replaced may be determined by fluoroscopy in combination with the use of a fluid contrast material to inflate the fluid expandable balloon.

10. The delivery catheter of claim 8, wherein the mean diameter of an aperture defined by a dilated valve to be replaced may be determined by fluoroscopy in combination with radiopaque markers distributed about a circumference of the fluid expandable balloon.

11. The delivery catheter of claim 10, wherein the radiopaque markers are disposed on a wall of the intermediate segment of the fluid expandable balloon.

12. The delivery catheter of claim 10, wherein the radiopaque markers are disposed within a wall of the intermediate segment of the fluid expandable balloon.

13. The delivery catheter of claim 8, wherein the mean diameter of an aperture defined by a dilated valve to be replaced may be determined from a combination of a volume of fluid delivered to the fluid expandable balloon and a pressure within the fluid expandable balloon resulting from the delivery of that volume of fluid to the fluid expandable balloon.

14. The delivery catheter of any of claims 1 through 13, wherein the deployable filter element located downstream of the fluid expandable balloon is configured and adapted to be placed in the second deployed configuration while the fluid expandable balloon is positioned with the intermediate segment generally centered within the dilated valve to be replaced and while the fluid expandable balloon is first inflated and then deflated.

15. A method for percutaneous valvuloplasty and heart valve replacement comprising:
inserting percutaneously a delivery catheter comprising an elongated tubular element having a proximal end, a distal end, and at least one lumen therebetween, a fluid expandable balloon, a deployable filter element, a replacement heart valve,
wherein the deployable filter element is located downstream of the fluid expandable balloon and has a first collapsed configuration and a second deployed configuration which filters substantially all blood passing the deployable filter element;
and wherein the delivery catheter further includes components which are configured and adapted to position and install a replacement heart valve;
advancing the delivery catheter until the fluid expandable balloon is positioned within a heart valve to be replaced;
actuating the deployable filter element from the first collapsed configuration to the second deployed configuration;
expanding the fluid expandable balloon within the heart valve to be replaced thereby performing a valvuloplasty;
collapsing the fluid expandable balloon;
actuating the deployable filter element from the second deployed configuration to the first collapsed configuration thereby containing any collected debris within the deployable filter element;
retracting the delivery catheter to remove the fluid expandable balloon from the heart valve to be replaced;
advancing the replacement heart valve and components which position and install a replacement heart valve from the distal end of the delivery catheter;
installing the replacement heart valve;
retracting the components which are configured and adapted to position and install the replacement heart valve within the distal end of the delivery catheter; and
withdrawing the delivery catheter.

16. The method of claim 15, further comprising between a step of expanding the fluid expandable balloon within the heart valve to be replaced and a step of collapsing the fluid expandable balloon inserting a step of determining at least one distance within a patient.

17. The method of claim 15, further comprising between a step of retracting the delivery catheter to remove the fluid expandable balloon from the heart valve to be replaced and a step of advancing the replacement heart valve and components which position and install a replacement heart valve inserting a step of actuating the deployable filter element from the first collapsed configuration to a second deployed configuration.

18. The method of claim 17, further comprising between a step of retracting the components which position and install the replacement heart valve within the distal end of the delivery catheter and a step of withdrawing the delivery catheter inserting a step of actuating the deployable filter element from the second deployed configuration to the first collapsed configuration thereby containing any collected debris within the deployable filter element.
